(19) United States
(12) Patent Application Publication
(10) Pub. No.: US 2008/0091225 A1
(43) Pub. Date: Apr. 17, 2008

(54) COUNTING DEVICE AND METHOD

(76) Inventors: John P. Cole, Alpharetta, GA (US); Tesfaye H. Gutema, Alpharetta, GA (US)

Correspondence Address: Berkeley Law and Technology Group, LLC, Suite 240, 1700 NW 167th, Beaverton, OR, 97006, US

(21) Appl. No.: 11/549,942

(22) Filed: Oct. 16, 2006

(51) Int. Cl. A61B 17/00; A61B 17/06; A61B 17/32

(52) U.S. Cl. 606/172; 606/1; 606/167; 606/184; 606/222

(57) ABSTRACT

In accordance with one or more embodiments, a Counting Device Assembly is capable of counting a number of times a surgical implement causes an incision during a surgical procedure.

### BACKGROUND

During a follicular transplant procedure a number of incisions are made in the scalp of a patient for receiving a hair follicle graft. The incisions may comprise a variety of shapes, such as, for instance, holes, curved apertures or straight cuts. Typically, the surgeon keeps track of the number of incisions for a variety of reasons. For instance, tracking the number of incisions is helpful to ensure that there are enough recipient incisions for receiving all of the follicular grafts a surgeon has taken from a donor site for transplanting into the scalp. Also, tracking the number of incisions is helpful for calculating the density of the hair in a recipient area after the procedure is complete. Additionally, customarily the cost of a hair transplant procedure may be based on the number a graft transplants made. Therefore, it may be helpful to know the number of incisions made in order to calculate the cost of the procedure. Current practice involves having either the person making the incisions or a technician physically count and track the number of incisions made. This may be tedious and distracts the surgeon while performing the procedure.

### BRIEF DESCRIPTION OF THE DRAWINGS

FIG. 1a is an exploded view of a particular embodiment of a Counting Device Assembly.

FIG. 1b is a front view of an assembly of the exploded components of a particular embodiment of a Counting Device Assembly as depicted in FIG. 1a.

FIG. 1c is a side section view of an assembly of the exploded components of a particular embodiment of a Counting Device Assembly as depicted in FIG. 1a.

FIG. 2a is a diagram of a blade hub and blade assembled for use in a particular embodiment of a Counting Device Assembly.

FIG. 2b is a side view of a blade hub and blade assembled for use in a particular embodiment of a Counting Device Assembly.

FIG. 2c is a diagram of a blade hub and blade assembled for use in a particular embodiment of a Counting Device Assembly.

FIG. 2d is a side view of a blade hub and blade assembled for use in a Counting Device Assembly.

FIG. 3 illustrates a particular embodiment of a Counting Device Assembly comprising a solenoid actuation mechanism, punch hub and tubular punch.

FIG. 4 illustrates a particular embodiment of a Counting Device Assembly comprising an actuator concentrically disposed about a hub.

### DETAILED DESCRIPTION

In the following detailed description, numerous specific details are set forth in order to provide a thorough understanding of claimed subject matter. However, it will be understood by those skilled in the art that claimed subject matter may be practiced without these specific details. In other instances, well-known methods, procedures, and components have not been described in detail so as not to obscure claimed subject matter.

The following disclosure relates generally to a counting device assembly and method. Although the examples and embodiments described herein refer to incisions made during a follicular transplant procedure, such examples are meant for illustrative purposes and are not intended to limit the scope of the disclosure. The disclosed device and method may be useful for counting a variety of procedures. Such procedures may include incision making, graft harvesting, marking and quantifying in hair transplantation, marking and quantifying skin lesions in dermatology and other applications. Additionally, the disclosed subject matter may be useful in non-surgical procedures such as marking, quantification, injections and vaccinations in veterinary medicine, tattooing, marking and/or quantifying in industrial applications or any other application. However, these are merely examples of a variety of procedures that may be counted by a counting device assembly and claimed subject matter is not limited in this regard.

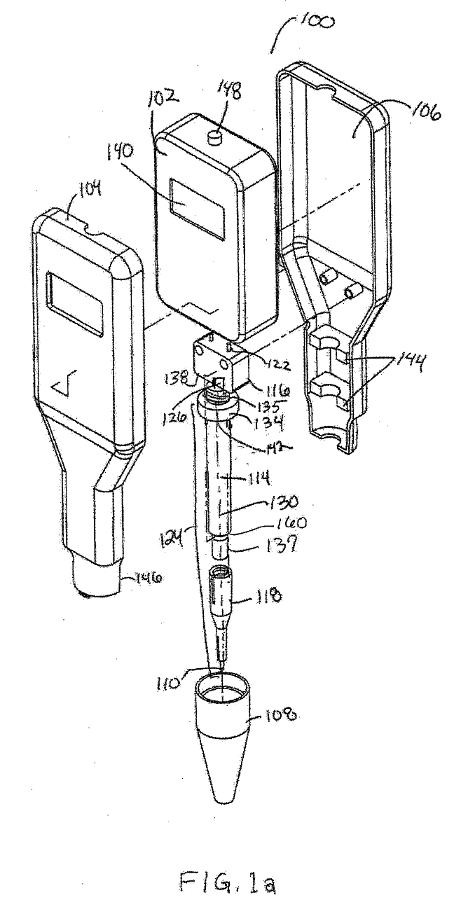

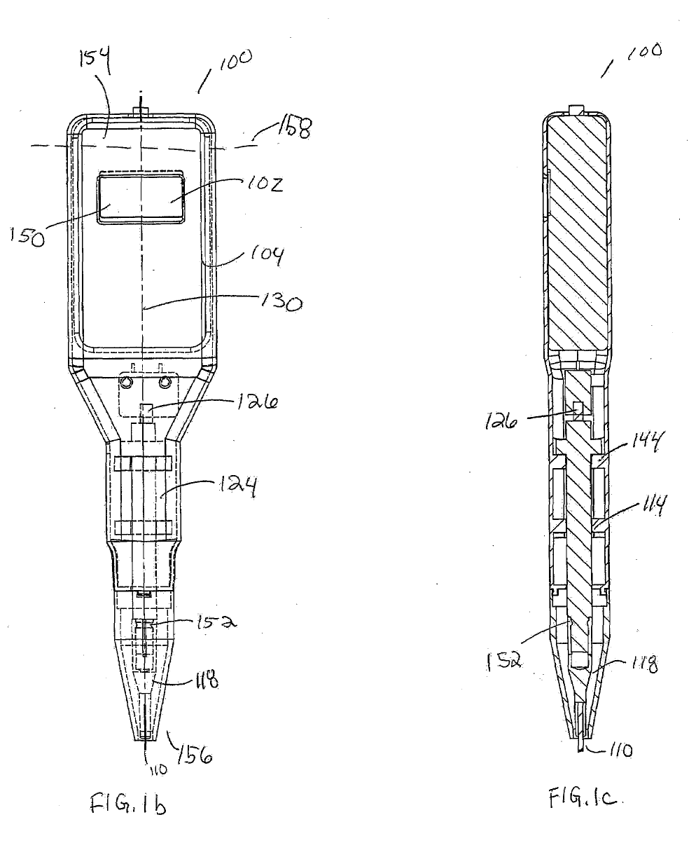

FIG. 1a illustrates an exploded view of a particular embodiment of a Counting Device Assembly (CDA) 100 comprising a counter 102 and a micro-switch 116. In a particular embodiment, CDA 100 may comprise a blade 110 and may be used for making incisions into the skin of a patient during a follicular transplant procedure. According to a particular embodiment, counter 102 may count the number of incisions made during the procedure thus enabling the surgeon and technicians to concentrate on other aspects of the procedure. During a follicular transplant procedure, an incision count may be recorded automatically at the time an incision is made. However, this is merely an example of a procedure that may be counted by a CDA and claimed subject matter is not limited in this regard.

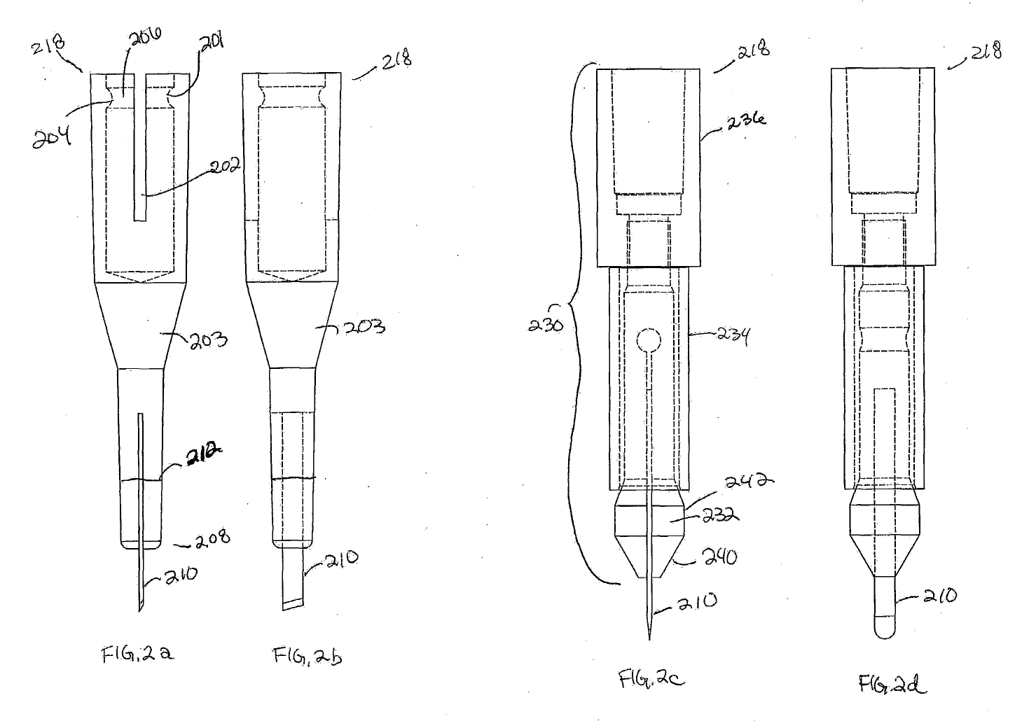

In a particular embodiment, CDA 100 may comprise an implement assembly 124. In a particular embodiment, implement assembly 124 may comprise actuator 114 assembled to blade hub 118 and blade 110. According to a particular embodiment, actuator 114 may be directly coupled to blade hub 118 and blade 110. In a particular embodiment, actuator 114 may comprise a projection 134 at first end 135 that may be capable of coming into contact with guides 144 of upper housing 104 and lower housing 106. At second end 137, actuator 114 may comprise a geometry that allows assembly of implements such as blades, needles, punches and/or markers, such as, for instance by, a luer connection, a snap-in and/or sliding friction fit. In a particular embodiment, blade hub 118 may be any of a variety of blade hubs, such as, for instance, any of the variety of blade hubs described with respect to in FIGS. 2a-2d. According to a particular embodiment, implement assembly 124 may comprise any of a variety of implements and hubs assembled to actuator 114. Such hubs and implements may comprise, for instance, a marker hub and marker, a needle hub and needle and/or a punch hub and punch. In another embodiment, an implement such as a blade 110 may be directly coupled to an actuator 114 without a hub by gluing, molding-in, threading and/or welding. However, these are merely examples of implement assemblies that a CDA may comprise and methods of coupling various implements with hubs and claimed subject matter is not limited in this regard.

During a hair transplant procedure, according to a particular embodiment, while the surgeon is making incisions into the scalp of the patient, CDA 100 may count and/or record the number of incisions. Each time a surgeon makes an incision using CDA 100, before blade 110 enters the skin, blade 110 may encounter a resistance force when pressed against the skin. The resistance force may cause implement assembly 124 to be displaced or retracted about 1 mm along a longitudinal length 130 of CDA 100. However, this is merely an example of a length and direction that a blade assembly may be displaced within a CDA and claimed subject matter is not limited in this regard.

In a particular embodiment, micro-switch 116 may be disposed above activation pin 126 of implement assembly 124. According to a particular embodiment, implement assembly 124 may come into contact with micro-switch 116 as implement assembly 124 is displaced. Such displacement may in turn move micro-switch contact arm 122 from an open to a closed position or from a off to an on position and may thus initiate counting. In a particular embodiment, retraction of implement assembly 124, as described above, may move contact arm 122 on micro-switch 116. According to a particular embodiment, micro-switch 116 may be electrically connected to an electronic counter (not shown). When the switch contacts are turned on, a current can flow into the circuit and counting may be performed by a digital circuit (not shown). When micro-switch is turned off, current is interrupted and no counting may be performed.

According to another particular embodiment, activation pin 126 may be made of an electrically conductive material, such as, for instance, copper and/or aluminum. According to a particular embodiment, when implement assembly 124 is retracted along longitudinal axis 130 about 1 mm, activation pin 126 of assembly 124 may come into direct contact with an open counting circuit (not shown) of micro-switch 116. According to a particular embodiment, when activation pin 126 comes into contact with micro-switch 116, activation pin 126 may activate micro-switch 116 by completing a counting circuit (not shown). According to a particular embodiment, micro-switch 116 may be electrically coupled to an electronic counter 102. Thus, with each displacement of implement assembly 124 of CDA 100, counter 102 may count and/or track an additional incision. In other embodiments, implement assembly 124 or actuator 114 may drive a mechanical counter arm (not shown) to initiate counting. However, these are merely examples of methods of activating a counter in a CDA and claimed subject matter is not limited in this regard.

In a particular embodiment, during a follicular transplant procedure, an incision count may be displayed in display window 140 adding one unit for every incision made or adding multiple units for every incision made, for example, in a particular embodiment, multiple blades may be attached to the end of the device. According to a particular embodiment, display window 140 may comprise a variety of displays, such as, for instance, an electronic and/or mechanical display. However, these are merely examples of the variety of displays that a CDA may comprise and claimed subject matter is not limited in this regard. Additionally, in a particular embodiment, counter 102 may comprise a power switch 148 capable of turning power on or off and/or capable of resetting counter 102 to zero. Counter 102 may be digital and may consist of an electronic board (not shown), a speaker/buzzer (not shown) and battery (not shown). In an alternative embodiment, counter 102 may be mechanical. The counting may be performed by adding one or two or more digits at a time. Such a mechanical counter may comprise a number of components and be driven by gears and/or belts. A mechanical counter may comprise an actuator that may be capable of moving a gear. As a gear rotates other gears at a given proportion, numbers written on the sides of the gears may be read in a display window.

According to a particular embodiment, micro-switch 116 may comprise a compression spring 142 that may be compressed by activation pin 126 against micro-switch 116. During a follicular transplant procedure (as described above), compression of compression spring 142 may occur when implement assembly 124 is pressed against the skin of a patient exerting a linear compression force on compression spring 142 prior to an incision. After an incision is made, blade 110 (part of implement assembly 124) may be extracted from the skin releasing pressure on compression spring 142, the linear restoring force of compression spring 142 may extend implement assembly 124 back to an original position. In another particular embodiment, a spring (not shown) may be coupled to activation pin 126 or may be incorporated within micro-switch 116. This is merely an example of a mechanical method of returning an implement assembly 124 to an original position after manual displacement. An implement assembly may be displaced and/or returned to an original position by a variety of methods such as, for instance, a manual displacement and/or return, an electronic displacement and/or return mechanism, a hydraulic displacement and/or return mechanism and/or a pneumatic displacement and/or return mechanism or some combination thereof. However, these are merely examples of the variety of displacement and/or return mechanisms that may be used to displace and/or return an implement assembly to an original position and claimed subject matter is not limited in this regard.

Referring still to FIG. 1, in a particular embodiment, activation pin 126 may be disposed above a projection 134 on actuator 114. According to a particular embodiment, implement assembly 124 may be displaced and/or retracted a particular length along the longitudinal axis 130. According to a particular embodiment, projection 134 may come into contact with a bottom surface 138 of micro-switch 116 in one direction or actuator guide 144 in the other direction substantially preventing axial displacement of implement assembly 124 beyond a particular length. Such a “particular length” may be about 1 mm. However, this is merely an example of a method of preventing axial displacement of an implement assembly beyond a particular length and claimed subject matter is not limited in this regard.

Various components of a Counting Device Assembly may be made by a variety of processes, such as, for instance, by molding, machining, stamping, casting or similar processes and may comprise features such as holes, grooves, bosses, ribs, and/or hooks capable of enabling assembly and guiding of various parts. Molded parts may be made of a variety of polymeric materials, such as, for instance, ABS, polycarbonate, acrylic, polypropylene and/or polyethylene. Machined or stamped parts may be made of a variety of materials, such as, for instance, plastic, stainless steel, copper, titanium, and/or aluminum. Additionally, protective painting or coating may be applied to portions of the disclosed device or to the whole device. Certain parts, such as, for instance, upper and lower housings may be assembled together by a variety of methods, such as, for instance, welding, gluing, snapping or by means of screws and/or hook and groove assemblies. However, these are merely examples of methods of manufacturing and assembly of a counting device assembly and claimed subject matter is not limited in this regard.

In a particular embodiment, CDA 100 may comprise an upper housing 104, lower housing 106 and tip cover 108. According to a particular embodiment, upper housing 104 and lower housing 106 may be assembled together and may enclose counter 102, micro-switch 116 and implement assembly 124. In a particular embodiment, upper housing 104 and lower housing 106 may be assembled together by a variety of methods, such as, for instance, welding, gluing, snapping and/or with mechanical connections such as screws. Micro-switch 116 and/or counter 102 may be mounted within upper housing and/or lower housing by a variety of methods, such as, for instance, with mounting screws, gluing, welding or snapping in. In a particular embodiment, upper housing 104 and lower housing 106 may comprise features such as guides 144 capable of guiding implement assembly 124 along longitudinal axis 130. Guides 144 may be coupled to implement assembly 124 with a sliding fit. The term sliding fit used here is intended to describe a fit between at least two members that allows a degree of independent motion. The strength of a sliding fit may fall anywhere on a continuum from loose to tight. However, this is merely an example of the variety of features that an upper housing and lower housing may comprise. Many other features such as holes, grooves, bosses, ribs, and/or hooks may enable assembly and guiding of different parts in a CDA and claimed subject matter is not limited in this regard.

In a particular embodiment, tip cover 108 may be assembled to upper housing 104 and lower housing 106 of CDA 100 by a removable assembling process, such as, for instance, a hook 146 and a groove (not shown). According to a particular embodiment, tip cover 108 may protect a user's fingers during use of CDA 100 and may be removed when changing implements such as, for instance, blades, needles and/or markers. However, this is merely an example of a tip cover for a CDA and assembling method thereof and claimed subject matter is not limited in this regard.

FIGS. 1b and 1c illustrate an assembly of the exploded components of a particular embodiment of a Counting Device Assembly (CDA) 100.

FIG. 1b shows a front view of a particular embodiment of a CDA 100. According to a particular embodiment, CDA 100 may comprise an upper housing 104 having a window 150 through which counter display 140 may be viewed. In a particular embodiment, a pin 126 may be disposed within micro-switch 116. In a particular embodiment, upper housing 104 and lower housing (not shown) may be coupled at a midline (not shown) with respect to a longitudinal axis 130. Additionally, according to a particular embodiment, upper housing 104 and lower housing (not shown) may comprise a wider profile with respect to a latitudinal axis 158 at first end 152 than at second end 156. However, this is merely an example of a shape and connection point for an upper and lower housing of a CODA and claimed subject matter is not limited in this regard.

FIG. 1c depicts a side view of a particular embodiment of a CDA 100. According to a particular embodiment, blade hub lock 152 may be locked into position securing blade 110, blade hub 118 and actuator 114. In a particular embodiment, implement assembly 124 may comprise blade hub lock 152, blade 110, blade hub 118 and actuator 114. Additionally, actuator 114 may be disposed within guides 144. However, this is merely an example of a method of securing and positioning components of an implement assembly. There may be a variety of methods of securing a blade hub to a blade and actuator, such as, for instance, by gluing, molding-in, using a pin vise assembly, and/or welding and claimed subject matter is not limited in this regard.

FIGS. 2a-2d depict a blade hub 218 and blade 210 assembled by various methods for use in a CDA. In a particular embodiment, for hair transplant procedures, blade 210 may have a thickness varying from about 0.05 mm to 0.35 mm with a thickness in one or more embodiments being about 0.1 mm to 0.25 mm, the width of blade 210 may be about 0.5 mm to 3 mm with in one or more embodiments being about 1.1 mm for body hair, 1.3 mm for scalp hair and 1.4 mm to 1.5 mm for strip hair transplant procedures. According to a particular embodiment, the thickness of blade 210 may be selected for a smaller, the smallest, or nearly smallest, functional value to minimize or reduce scar size and reduce trauma, excessive bleeding, and graft compression. Blade 210 may have a variety of profiles such as an inclined edge and/or curved or arced edges as shown in FIG. 2b and FIG. 2d. Such profiles may help to reduce trauma, reduce the risk of damaging tissue at the bottom end of the incision, and/or reduce the required force to make the incision, minimizing or reducing the risk of damaging tissue or blood vessels. However, these are merely examples of dimensions a blade in a CDA may have and claimed subject matter is not limited in this regard.

FIG. 2a, illustrates a particular embodiment of blade hub 218 and blade 210 assembled together by molding-in or gluing. In a particular embodiment, blade hub 218? may be made of a variety of materials and by a variety of processes as described above with respect to FIG. 1a. According to a particular embodiment, at a first end 206, blade hub 218 may comprise, an inner cavity 202 comprising a lip 204. According to a particular embodiment, lip 204 may mate to a groove 160 on actuator 114 as shown in FIG. 1a. According to a particular embodiment, second end 208 may comprise a second groove 210 capable of coupling an implement such as blade 210 to blade hub 218. According to a particular embodiment, blade 210 and blade hub 218 may be securely coupled by molding-in and/or gluing. However, these are merely examples of methods by which a blade and blade hub may be coupled and claimed subject matter is not limited in this regard. FIG. 2b illustrates a side view of blade hub 218 and blade 210 depicted in FIG. 2a to illustrate a profile view of blade 210.

FIG. 2c illustrates a particular embodiment of a blade hub 218 and blade 210 assembled together by a pin-vice mechanism. In a particular embodiment, blade 210 may be coupled to a blade hub 218 by pin-vice 230. Pin-vice 230 may consist of collet 232, sleeve 234 and adaptor nut 236. According to a particular embodiment, adaptor nut 236 may be threaded and coupled to sleeve 234 by mating threads (not shown) on sleeve 234. According to a particular embodiment, adaptor nut 236 may be tightened against sleeve 234 thus compressing tapered end 240 of cone 242 and tightening against blade 210 placed in the center of collet 232. FIG. 2d illustrates a side view of blade hub 218 and blade 210 depicted in FIG. 2c to illustrate a profile view of blade 210. Pin vise 230 blade 210 may be assembled to an actuator tip (not shown) by a variety of ways, for example by friction fit, luer lock, threading or snap-in. However, these are merely examples of different methods of coupling an implement to a hub and actuator for use in a CDA and claimed subject matter is not limited in this regard.

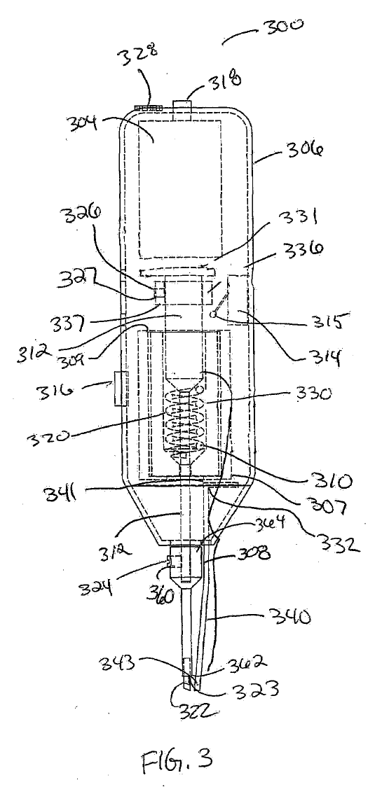

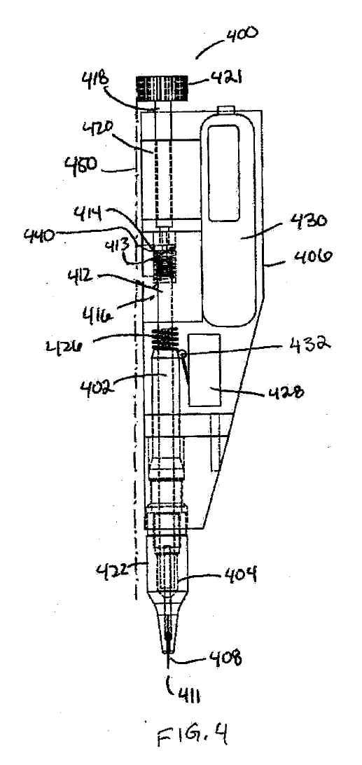

FIG. 3a, illustrates the assembly of a particular embodiment of CDA 300 comprising a solenoid actuation mechanism, punch hub 308 and tubular punch 322. During a follicular hair transplant procedure a CDA 300 comprising a tubular punch 322 may be used by a surgeon to remove hair follicles from the skin of a patient by inserting punch 322 a particular depth into the skin surrounding a hair follicle and then extracting the skin and hair follicle using punch 322.

In a particular embodiment, CDA 300 comprises housing 306, tubular punch hub 308, solenoid coil 310, actuator 312, a micro-switch 314, on-off switch 318, counter 304, actuator return spring 320, solenoid operating switch 316, tubular punch 322, first set screw 324, second set screw 326 and power input connection 328. According to a particular embodiment, CDA 300 is assembled similarly to CDA 100 described with reference to FIG. 1a. As described above, micro-switch 314, counter 304, actuator 312 and punch hub 308 (similar to blade hub described above) may be encased in housing 306. Additionally, in a particular embodiment, solenoid coil 310, battery or power adaptor (not shown) and actuator return spring 320, may also be encased in housing 306. According to a particular embodiment, punch hub 308 may couple actuator 312 and tubular punch 322. Assembly of punch hub 308, actuator 312 and tubular punch 322 may be referred to as implement assembly 332. However, these are merely examples of various parts that may be assembled in a CDA and claimed subject matter is not limited in this regard.

In a particular embodiment, solenoid 330 may be activated by an AC or DC voltage applied to solenoid coil 310. According to a particular embodiment, a DC source, such as a battery (not shown) may be assembled within housing 306. However an external power source may be connected to solenoid coil 310 via power input connection 328. In a particular embodiment, when electric current is applied to solenoid coil 310 it may produce an electromagnetic force that may act on actuator 312. This force may extend or retract actuator 312 along longitudinal axis 331 of CDA 300 a particular distance. Such a distance may be for instance about 1 mm to 5 mm or larger. In a particular embodiment, displacement of actuator 312 may displace punch hub 308 and tubular punch 322 an equal distance. According to a particular embodiment, actuator return spring 320 may be disposed within solenoid 330 and coupled to actuator 312. After solenoid 330 has displaced actuator 312 a particular distance, actuator return spring 320 may return actuator 312 to an original position. In a particular embodiment, solenoid operating switch 316 may activate solenoid 330. When solenoid 330 is activated, actuator 312 may be displaced with respect to longitudinal axis 331 a particular distance. According to a particular embodiment, actuator 312 may not return to an original position as long as switch 316 is in an “on” state. In a particular embodiment, when switch 316 is released, actuator 312 may be reset back to an original position by actuator return spring 320. However, this is merely an example of a method of displacing an actuator within a CDA and claimed subject matter is not limited in this regard. There may be a variety of other methods of displacing an actuator such as, for instance, with a threaded screw attachment, and/or a hydraulic, pneumatic and/or a manual displacement mechanism.

According to a particular embodiment, a first set screw 324 may enable assembly of punch hub 308, actuator 312 and tubular punch 322. In a particular embodiment, actuator 312 may be seated with first end of punch hub 364. First set screw 324 may mate to a threaded bore hole 360 on punch hub 308 and contact actuator 312. Tubular punch 322 may be seated with a glued connection, threaded or friction fit within second end 362 of punch hub 308. However, this is merely an example of a method of securing a punch and actuator to a hub and claimed subject matter is not limited in this regard.

Additionally, according to a particular embodiment, depth adjust ring 336 may be positioned along longitudinal axis 331 and may be fixed in position with a second set-screw 326. Depth adjust ring 336 may limit extension of actuator 312 along longitudinal axis 331 to a desired length thereby limiting the extension of punch 322 from tip end 323 of CDA 300. Depth adjust ring may prohibit extension of any of a variety of an implements coupled to actuator 312, such as, for instance, a blade, a needle or marker. Other methods of limiting displacement may include disposing a projection on actuator 312 that may contact a guide (not shown) on an inside surface of housing 306. Another method of limiting displacement of an implement may be coupling a surface such as a lip to an implement such as punch 322 limiting extension of punch 322 into the skin of a patient beyond the lip. However, these are merely examples of methods of limiting the displacement of an implement and/or actuator in a CDA and claimed subject matter is not limited in this regard.

In a particular embodiment, depth adjust ring 336 may also operate micro-switch 314. As actuator 312 is displaced or extended, a first side 337 of depth adjust ring 336 may push lever switch 315 of micro-switch 314 thereby closing the circuit of counter 304 to initiate counting. In a particular embodiment, micros-switch 314 may be electronically coupled to counter 304. Counter switch 318 may turn counter 304 on or off and may reset counter 304 to zero. However, this is merely an example of a method of activating and deactivating a counter and claimed subject matter is not limited in this regard.

In a particular embodiment, at a first end 341, a zero reference edge (probe) 340 may be coupled to housing 306 and/or to solenoid housing 307. At second end 343, zero reference edge 340 may be aligned with an implement in a retracted or zero position at tip end 323 of CDA 300. In a particular embodiment, such an implement may be a punch 322. According to a particular embodiment, during a follicular extraction, zero reference edge 340 may enable alignment of punch 322 with the skin surface at the follicular extraction site. During the procedure, zero reference edge 340 may lightly contact the surface of the skin. Here, zero reference edge 340 may establish an incision starting position by touching the surface of the skin. Punch 322 then may be thrust out beyond zero reference edge 340 to a given depth.

According to a particular embodiment, the depth punch 322 may extend into the skin of a patient and may be limited by a depth adjust nut 336 located on actuator 312 under shoulder 337. Depth adjust nut 336 may be secured to actuator 312 by second set screw 326 through a threaded bore hole 327. In a particular embodiment, as solenoid 316 extend actuator 312, depth adjust nut 336 may come into contact with top side 309 of solenoid 316 preventing further extension of actuator 312. However, this is merely an example of preventing extension of a punch in a CDA beyond a certain depth and claimed subject matter is not limited in this regard.

According to a particular embodiment, solenoid switch 316 may be turned on to activate solenoid coil 310. In a particular embodiment, solenoid coil 310 may quickly displace punch 322. This movement may incise the skin surrounding a hair follicle at a specific angle and specific depth. According to another embodiment, rather than a punch 322 a blade or other implement may be used. In an embodiment utilizing a blade, a consistent force and rate of application of the incision procedure may be less traumatic to the skin of a patient than a manual method of incision.

FIG. 4 illustrates an assembly of a particular embodiment of a CDA 400 comprising an actuator 402 concentrically disposed about blade hub 404. In a particular embodiment, depth adjust screw 418 may adjust the length of a blade 408 extending into the skin of a patient. According to a particular embodiment, depth adjust screw 418 and depth adjust pin 412 may be coupled by a pin return spring 414. According to a particular embodiment, depth adjust pin 412 may comprise pin shoulder 413. Pin return spring 414 may be housed between pin shoulder 413 and spring box 416. In a particular embodiment, pin return spring 414 may be compressed when depth adjust pin 412 is displaced a particular distance and capable of returning depth adjust pin 412 to an original position after displacement. According to a particular embodiment, depth adjust screw 418 may be threaded to housing 406 at threaded guide 420 and may be capable of adjusting the depth to which blade 408 may be inserted into the skin of a patient. According to a particular embodiment, depth adjust pin 412 may extend down through housing 406 and may be coupled to blade hub 404 by a variety of coupling methods such as threading, molding-in and/or gluing. As depth adjust screw 418 is rotated clockwise by handle 421, it may displace depth adjust pin 412, blade hub 404 and blade 408 a particular length along a longitudinal axis 450 and may compress pin return spring 414. The blade depth is therefore determined by the length protruding out of the actuator tip plus the actuator displacement length. In a particular embodiment, a depth indicator 440 may be attached to the depth adjust pin 412. Blade depth may be set using the scale indicator. However, this is merely an example of a method of adjusting the depth of a blade in a CDA and claimed subject matter is not limited in this regard. In a particular embodiment, actuator 402 may activate micro-switch 428 to initiate counting. According to a particular embodiment, actuator 402 may be coupled to actuator tip 422 by a variety of removable coupling methods such as threading. Micro-switch 428 may be coupled electronically to a counter 430. Actuator 402 may slide along longitudinal axis 450 over depth adjust pin 412. When blade 408 is inserted in to the skin, at the end of an incision stroke, actuator tip 422 may be pushed against the skin causing actuator 402 to retract against return spring 426. At the same time actuator 402 may push micro-switch lever 432 of the micro-switch 428 to initiate counting. Once the counting is done, blade 408 may be retracted. At this time, the return spring restores actuator 402 back to an original position. The force exerted on the actuator may be generated by other means like mechanical, pneumatic and hydraulic systems or may be generated by the procedure like dissection, incision or marking. However, this is merely an example of a method of activating a micro-switch and claimed subject matter is not limited in this regard.

In a particular embodiment, depth adjust pin 412 may comprise a depth indicator 440 which may indicate the depth a blade 408 may be inserted into the skin of a patient. Depth indicator 440 may be viewed through an indicator window (not shown) on the side of CDA 400. Depth indicator 440 indicates depths by moving along a printed scale (not shown) along the side of CDA 400. As depth adjust screw 418 is screwed into housing 406 depth adjust screw 418 may move the indicator because depth adjust screw 418 may be coupled to depth adjust pin 412. Depth adjust pin 412 may be securely coupled to blade 408 and blade hub 404 and may extend blade 408 beyond tip 411 of CDA 400 an amount corresponding to the depth to which depth adjust screw 418 has been adjusted. Thus as depth adjust screw 418 changes the depth blade 408 may enter the skin the depth may be viewed in the depth indicator window (not shown). However, this is merely an example of a method of indicating a depth adjustment and claimed subject matter is not limited in this regard.

While certain features of claimed subject matter have been illustrated as described herein, many modifications, substitutions, changes and equivalents will now occur to those skilled in the art. It is, therefore, to be understood that the appended claims are intended to cover all such embodiments and changes as fall within the true spirit of claimed subject matter.

What is claimed is:

1. An apparatus, comprising:
a housing;
an actuator having a surgical implement disposed at one end and being disposed within the housing; and
a counter disposed in the housing;
wherein the surgical implement is capable of at least making contact with a body tissue during a procedure and wherein the counter is capable of registering a count in response to the actuator being actuated to count at least making contact with the body tissue.

2. An apparatus according to claim 1, wherein the counter is capable of accumulating a number of at least making contact with the body tissue for a procedure.

3. An apparatus according to claim 2, the counter further comprising a display capable of displaying a count.

4. An apparatus according to claim 3, wherein the counter is disposed within the housing, the housing having a window formed thereon through which a count of the counter may be viewed.

5. An apparatus according to claim 1, wherein the implement comprises one or more of the following: at least one blade, at least one punch, at least one needle, or at least one marker, or combinations thereof.

6. An apparatus according to claim 1, wherein the actuator is capable of being displaced with respect to an original position in response to one or more of the following: activation of at least one solenoid coil, manual force, mechanical force, hydraulic force, pneumatic force, or electromotive force, or combinations thereof.

7. An apparatus according to claim 1, wherein the actuator is capable of being displaced with respect to an original position, and further comprising a spring coupled to the actuator and capable of returning the actuator to the original position.

8. An apparatus according to claim 4, further comprising a depth indicator coupled to the housing.

9. An apparatus according to claim 1, further comprising a depth controller coupled to the actuator.

10. An apparatus according to claim 1, further comprising a depth controller coupled to the implement.

11. An apparatus according to claim 9, wherein the depth controller comprises one or more of the following: a shoulder, a stop, a perpendicular surface, a lip, a projection, an adjustment lever, or an adjustment screw, or combinations thereof.

12. An apparatus according to claim 10, wherein the depth controller comprises one or more of the following: a shoulder, a stop, a perpendicular surface, a lip, a projection, an adjustment lever, or an adjustment screw, or combinations thereof.

13. An apparatus according to claim 4, further comprising at least one guide disposed on an inside surface of the housing and capable of guiding the actuator along a longitudinal axis of the housing.

14. An apparatus according to claim 1, further comprising a hub capable of securing the implement to the actuator.

15. An apparatus according to claim 14, wherein the hub comprises a pin vice.

16. An apparatus according to claim 1, further comprising a depth adjustment pin coupled to the implement and capable of substantially controlling an extension of the implement.

17. An apparatus, comprising:
a housing;
an actuator having an implement disposed at one end and being disposed within the housing; and
a counter disposed in the housing;
wherein the actuator is capable of causing the implement to at least make contact with a surface in response to the actuator being actuated and the counter is capable of registering a count in response to the actuator being actuated to count at least making contact with the surface.

18. An apparatus according to claim 17, wherein the counter is capable of accumulating a number of at least making contact with the surface for a procedure.

19. An apparatus according to claim 18, wherein the counter is disposed within the housing, the housing having a window formed thereon through which a count of the counter may be viewed.

20. An apparatus according to claim 17, wherein the actuator is capable of being displaced with respect to an original position in response to one or more of the following: activation of at least one solenoid coil, manual force, mechanical force, hydraulic force, pneumatic force, or electromotive force, or combinations thereof.

21. An apparatus according to claim 17, wherein the actuator is capable of being displaced with respect to an original position, and further comprising a spring coupled to the actuator and capable of returning the actuator to the original position.

22. An apparatus according to claim 17, further comprising a depth controller coupled to the actuator.

23. An apparatus according to claim 22, wherein the depth controller comprises one or more of the following: a shoulder, a stop, a perpendicular surface, a lip, a projection, an adjustment lever, or an adjustment screw, or combinations thereof.

24. A method comprising:
displacing an implement to result in contact with a body tissue; and
counting the number of times the implement is displaced to count the number of contacts with a body tissue during a surgical procedure.

25. A method according to claim 24, further comprising;
recording the number of times the implement is displaced; and
displaying a message related to the number of displacements.

26. A method according to claim 24, further comprising replacing the implement back to an original position after a displacement.

27. A method according to claim 24, further comprising controlling a length of the displacement of the implement.

28. An apparatus comprising:
means for displacing an implement to result in a contact with a body tissue; and
means for counting the number of times the implement is displaced to count the number of contacts with a body tissue during a surgical procedure.

29. An apparatus according to claim 28, further comprising;
means for recording the number of times the implement is displaced; and
means for displaying a message related to the number of displacements.

30. An apparatus according to claim 28, further comprising means for replacing the implement back to an original position after a displacement.

31. An apparatus according to claim 28, further comprising means for controlling a length of the displacement of the implement.
